(19) United States
(12) Patent Application Publication
(10) Pub. No.: US 2002/0081729 A1
(43) Pub. Date: Jun. 27, 2002

(54) CONTROLLED RELEASE OF TISSUE CULTURE SUPPLEMENTS

(76) Inventors: MARTIN C. PETERS, ANN ARBOR, MI (US); JONATHON A. ROWLEY, ANN ARBOR, MI (US); DAVID J. MOONEY, DEXTER, MI (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/276,747

(22) Filed: Mar. 26, 1999

Related U.S. Application Data
(60) Provisional application No. 60/079,535, filed on Mar. 27, 1998.

(51) Int. Cl. C12N 5/00; C12N 5/02; A61K 9/14

(52) U.S. Cl. 435/406; 435/405; 435/404; 424/488; 424/493

(57) ABSTRACT

The materials and methods are particularly useful in media for cell culturing but have other applications as well, such as in tissue engineering. The materials comprise at least one growth factor or other cell growth facilitating substance contained within a polymeric structure which can incorporate the substance and allow controlled release of the substance. Alginate materials are particularly useful as the polymeric structure. Another feature described herein is the above-described materials provided in a lyophilized (freeze-dried) form which greatly enhances their storage life.

Described herein are materials and methods for the controlled delivery of growth factors and other materials which facilitate the growth of cells. The materials and methods are particularly useful in media for cell culturing but have other applications as well, such as in tissue engineering. The materials comprise at least one growth factor or other cell growth facilitating substance contained within a polymeric structure allows controlled release of the substance. Alginate materials are particularly useful as the polymeric structure. Another feature described herein is the above-described materials provided in a lyophilized (freeze-dried) form which greatly enhances their storage life.

### BACKGROUND OF THE INVENTION

As the field of cellular biology continues to grow, the need for better defined systems for cell culture is ever increasing. Recently, interest has come to focus on defined media for cell culture. A defined media is one in which the exact amount of every component which makes up the media is known (e.g. amino acids, growth factors, hormones, sugars, salts, etc.). Currently most cell culture media contain 2-20% blood serum (generally bovine in origin) which contain undefined quantities of proteins and hormones. Cells tend to grow well in the presence of serum, which accounts for its use in cell culture. But serum also acts as a confounding factor when one wishes to study the effects of particular proteins and hormones or particular amounts thereof on cells, since the specific components and amounts in the serum are generally not known. In the last few years defined media for the culture of most cell types have found their way to market. These media have been a boon to many research projects but their high cost (˜$100 for 500 mL) when compared to serum-containing media (˜$25 for 500 mL) has made them a specialty item.

The interest in defined media has been heightened by the recent increase in the incidence of bovine spongiform encephalopathy (BSE). Materials derived from bovine tissue, which BSE is believed to be spread by, are widely used in cell culture products. Since humans are more susceptible to infection by BSE than originally thought, significant interest has developed around new cell culture products which are free of bovine materials. See, e.g., Narang H, Proc. Soc. Exp. Biol. Med., 211 (1996) 306-322; and Collinge J. et al., Nature, 383 (1996) 685-690. The new BSE awareness enhances interest in serum-free products.

In light of the need for defined serum-free media for both health and scientific reasons the market is expected to increase dramatically. See “Cell and Tissue Culture Market” in Genetic Engineering News, Jan. 1, 1998, p.8. The cost discrepancy between defined and serum-containing media can be attributed, in a large part, to the presence of protein growth factors (GF) (cost ˜$25 per microgram) in the defined media. Protein GF (e.g., basic fibroblast growth factor (bFGF), vascular endothelial growth factor (VEGF), epidermal growth factor (EGF), etc.) encourage cell survival and proliferation of cells but are subject to functional instability in the media.

Traditionally, defined media is made by simply adding the necessary supplements to a basic media formulation (e.g. Media 199, DMEM) in a stock bottle which is then used repeatedly for a finite period of time. The fact that GF degrade and generally lose effectiveness over time has led investigators to either add a significantly higher mass of GF to their media or add fresh GF daily. These strategies are expensive and labor intensive. Perfusion systems have also been developed which continuously feed cultures with new medium to maintain constant GF concentrations but these systems are even more costly.

### SUMMARY OF THE INVENTION

The invention provides a solution to the problem of the high cost of growth factors and similar substances in defined media by prolonging the life-time and effectiveness of the growth factors and/or other substances in the media so that less overall growth factor is required. The achievement of prolonging the life-time and effectiveness of the growth factors and/or other substances finds other applications as well, for example in tissue engineering. Another aspect of the invention also provides a means to maintain a constant concentration of active growth factor for extended times, i.e. storage stability.

Upon further study of the specification and appended claims, further objects and advantages of this invention will become apparent to those skilled in the art.

One embodiment of the invention is accordingly directed to materials which provide controlled release of growth factors (GF), and/or similar materials which facilitate the growth of cells, particularly in a quantifiable manner, to cell culture media which contain such materials and to processes for culturing cells using such material. The materials comprise at least one growth factor or other cell growth facilitating substance contained within a polymeric structure which incorporates the substance and allows controlled release of the substance. When used in tissue culture media, together with the other standard materials used in cell culture media (e.g. amino acids, growth factors, hormones, sugars, salts, etc.), the materials provide the cells which are being cultured with a constant amount of GF or similar substance. The materials can also be designed such that the controlled delivered amount is easily modulated to provide a release particularly suited to the application. Use of these materials means that less cell culture supplements are required to yield the same effect and that precise amounts of the growth agent can be delivered to the cells. Further, the need for serum-containing supplements is reduced or eliminated. By using the invention one can dose cell culture media only once and still insure predictable GF levels.

As the material for the polymeric structure, alginate materials are particularly useful, however, other polymeric materials are also useful. Also, blends of polymers including different types of alginates or alginates with other polymers are useful. In fact, almost any polymer or polymer form can be used for this application as long as it provides a controlled release effect of the growth factor or similar substance. The controlled release can be effected, for example, by degradation of the polymer structure when used and/or by providing a pore structure which allows for controlled diffusion of the growth factor or other substance. It is preferred that the polymer structure be selected such that the growth factor or similar substance be released over a period of 1 day to 3 months, particularly preferably 3 days to 1 month.

Materials which can provide such a polymeric structure include, for example, polylactic acid, polyglycolic acid, PLGA polymers, alginates and alginate derivatives, collagen, agarose, natural and synthetic polysaccharides, polyamino acids such as polypeptides particularly poly(lysine), polyesters such as polyhydroxybutyrate and poly-ε-caprolactone, polyanhydrides, polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids) and copolymers of the above, including graft copolymers.

The preferred materials are alginates. Alginate molecules are comprised of (1-4)-linked β-D-mannuronic acid (M units) and α-L-guluronic acid (G units) monomers which vary in proportion and sequential distribution along the polymer chain. Alginate polysaccharides are polyelectrolyte systems which have a strong affinity for divalent cations (e.g. Ca⁺², Mg⁺², Ba⁺²) and form stable hydrogels when exposed to these molecules. See Martinsen A., et al., Biotech. & Bioeng., 33 (1989) 79-89. Calcium cross-linked alginate hydrogels have been used in many biomedical applications, including materials for dental impressions (Hanks C. T., et al., Restorative Dental Materials; Craig, R. G., ed., Ninth Edition, Mosby (1993)), wound dressings (Matthew I. R. et al., Biomaterials, 16 (1995) 265-274), an injectable delivery medium for chondrocyte transplantation (Atala A., et al., J. Urology, 152 (1994) 641-643), and an immobilization matrix for living cells (Smidsrød O., et al, TIBTECH 8 (1990) 7 1-78).

Alginate has been previously utilized as a vehicle for controlled delivery of growth factors (Nugent M. A., et al., Mat. Res. Soc. Symp. Proc., 252 (1992) 273-284; and Downs M. L., et al., J. Cell. Phys., 152 (1992) 422-429). However, it has not been used as a vehicle for the controlled delivery of growth factors or similar substances in cell culture media. Further, previous alginate drug delivery technology has not adequately addressed long-term storage of incorporated cell culture supplements (e.g., growth factors).

According to the claimed invention, predictable drug release kinetics are achieved along with relatively high overall drug incorporation. Such control can be obtained for alginate materials, for example, by controlling the gelling properties of the alginate. The gelling properties of the alginate are controllable by, for example, varying the calcium or other divalent ion concentration and/or by modification of the structure of the alginate. Further, storage stability is addressed by the lyophilization embodiment of the invention discussed below.

Among the alginates useful in the invention for containing the growth factors or similar substances are included conventional alginate materials, such as described in the above publications, for example, and modified alginates. As modified alginates are included those which are modified from natural alginates as to their number and distribution of M units and G units to provide desired properties. Also, included are polymers which include alginate chains such as described in PCT application PCT/US97/16890 filed Sep. 19, 1997, which is incorporated herein by reference. For example, such materials may have alginate or modified alginate chains included as side chains or auxiliary chains from a backbone polymer chain, which backbone may also be an alginate or other polysaccharide. Further, the alginate chains may be crosslinked between side chains, auxiliary chains and/or backbone chains.

The invention can be applied to a variety of cell growth agents which include known growth factors (GF), including, for example, vascular endothelial growth factors, basic fibroblast growth factor, transforming growth factor beta, nerve growth factor, epidermal growth factor, platelet derived growth factor, etc., and other substances similar to GF which facilitate cell growth, particularly in cell culture media. Such other substances include hormones and other lipo-proteins and glyco-proteins.

The cell growth agents can be incorporated into the polymer structure in a manner which would be generally known in the art or determinable by one skilled in the art. One useful method is described in the examples below and schematically shown in FIG. 1. Therein, a solution of the growth agent is introduced into a solution of the polymer, e.g. alginate. The combined solutions are mixed, for example by vortexing. This mixed solution is then added dropwise, e.g. by passage through a syringe needle, into a solution which hardens the polymer, e.g. by gelling, polymerizing and/or cross-linking. In the case of an alginate polymer structure, the mixed solution may be dropped into a solution containing divalent cations, particularly Ca⁺² ions, which gel the alginate. The beads formed from the drops in this manner are then removed, e.g. by filtration. When using alginate material, it may also be desired to add SHMP, a gelling retardant, to the initial alginate solution to prevent premature gelling. The resulting beads are preferably generally spherical in shape. For cell culture media applications, the particles preferably have an average diameter of from 1 mm to 5 mm, more preferably from more than 1 mm to 4 mm, and for tissue engineering applications the particles preferably have an average diameter of from 10 nm to less than 1 mm. Processes providing a similar desired result are known in the art; some being described in the articles cited herein, for example. However, none of these processes describe the use of such beads for cell culture media. The concentration of cell growth agent within the particles can be varied within a wide range. Although not intended as a limitation, for most applications, the preferred practical concentration range is from about 10 ng to 1 μg of agent per particle or bead.

The above-described materials can be used in cell culture media in place of growth agents which may be added in a free form or some other form for defined media and/or in place of serum components. Thus, they are added together with the nutrients for the cell, e.g. amino acids, and, optionally, with other growth factors, hormones, sugars, salts, etc., which are required by the cell for growth or facilitation of growth. The other components of the cell culture media are well known or readily determinable by one of ordinary skill in the art depending upon the particular manner of cell growth desired. The amount of the beads added to the cell culture is dependent upon the amount of growth agent desired to be released. It is an advantage of the invention that, by designing the beads for a certain release rate and providing a certain amount of beads for a certain medium, precise control of the growth agent provided can be obtained. That is, the inventive materials provide advantageous controlled release of cell growth agents through the design of the polymeric structure to provide a quantifiable amount of cell growth agent over a certain time period. The release properties of the polymeric structure can be controlled, based on the agent being released, through the selection of a polymer structure and modifications thereof which effect its degradeability and porosity characteristics. Regarding alginates and modified alginates, means for modifying such characteristics are discussed, for example, in PCT application PCT/US97/16890, cited and incorporated above.

In another embodiment of the invention, established drug delivery technology is used in concert with a novel processing scheme to provide materials useful in cell culture media and other applications, such as tissue engineering. This embodiment of the invention utilizes polymer beads, particularly of alginate polysaccharide hydrogels, as described above as a delivery vehicle for growth factors and other cell culture media supplements. As above, these beads stabilize the growth factor from damaging environmental conditions and release it into the media in a controlled manner over time. However, they also exhibit a prolonged storage life, compared to similar hydrogel technology, due to the fact that they are lyophilized (freeze dried).

The lyophilization may be conducted in a conventional manner. For example, in a first step the isolated beads formed as described above are flash frozen, for example in liquid nitrogen or in a freezer at, preferably, −70° C. The freezing step is preferably conducted in a manner such that the rate of temperature change of the beads is at least 1° C. per minute, more preferably 5° C. per minute, so that the beads are frozen in a stabilized manner. Next, the frozen beads are subject to a vacuum such that the moisture is removed therefrom by sublimation. This can be accomplished in a standard lyophilization unit.

The lyophilized beads exhibit the advantageous property of being storage stable and transport stable. This stability can be maintained for up to 3 to 6 months and the beads then used with little or no loss in their effectiveness for controlled release of the growth agents. The lyophilized beads can be reconstituted for use by placing them in an aqueous solution or can be reconstituted in-situ when used in an aqueous environment. The lyophilized beads are useful in cell culture media in the manner described above. But they also find use in other applications where controlled delivery of cell growth agents is desired, such as in tissue engineering applications.

A particular tissue engineering application is for cells cultured in vitro on matrices which are then transplanted into a patient. Expansion of autologous cells in vitro offers the possibility of creating a tissue of substantial size from a small initial tissue biopsy. Matrices fabricated from synthetic or natural materials can be designed to provide a potential space in which the cells can proliferate and guide the formation of a new three-dimensional tissue. See Mooney, D. J., et al., Controlled Drug Delivery: Challenges and Strategies, Park, K., ed., pp. 333-346 (1997). A significant challenge in all cell transplantation endeavors is to improve upon the poor survival of most cell types shortly after they are implanted into the body (Mooney, D. J., et al., “Localized delivery of epidermal growth factor improves the survival of transplanted hepatocytes” Biotech. & Bioeng. 50:422-429 (1996)). A primary reason for this phenomenon is the lack of nutrients available to the transplanted cells (Colton, C. K., “Implantable biohybrid artificial organs” Cell Trans. 4:415-436 (1995)). A vascular network must be rapidly formed throughout the new tissue to ensure an adequate supply of essential nutrients (e.g., oxygen). This process of generating new microvasculature, termed angiogenesis, is a process observed physiologically in development and wound healing (Polverini, “The pathophysiology of angiogenesis” Crit. Rev. Oral. Biol. Med. 6:230-247 (1996)).

A variety of tissue-inducing substances (e.g., growth factors) that promote angiogenesis have been identified (Klagsbrun, M., et al., “Regulators of angiogenesis” Annu. Rev. Physiol. 53:217-239 (1991); and Polverini, supra), and they may accelerate the vascularization of transplanted cell/polymer matrices. These substances stimulate the appropriate cells (e.g. endothelial cells), already present in the patient's body, to migrate from the surrounding tissue, proliferate, and finally differentiate into blood vessels. Previous studies have demonstrated that it is possible to promote angiogenesis through the delivery of peptide growth factors such as basic fibroblast growth factor (bFGF) and vascular endothelial growth factor (VEGF) (Ingber et al., “Mechanochemical switching between growth and differentiation during fibroblast growth factor-stimulated angiogenesis in vitro: Role of extracellular matrix” J. Cell Biol. 109:317-330 (1989); Klagsbrun, supra,; Pepper et al., Biochem. and Biophys. Res. Comm., 189:824-831 (1992); and Goto et al., Lab. Invest. 69:508-517 (1993)).

The lyophilized materials described above, in addition to their advantageous use in cell culture media, are also advantageously used for promoting angiogenesis according to the above objectives with the added advantages of controlled release properties and storage stability of the growth factor in the polymeric structure. The alginate beads can be either seeded along with cells onto polymer matrices for tissue transplantation, incorporated directly into the bulk of matrices or implanted alone to promote tissue generation.

A further embodiment of the invention is a specific material which exhibits surprising and unexpected results making it particularly useful not only in cell culture media but, also, in other applications, such as the tissue engineering application described above. This embodiment focuses on the controlled delivery of vascular endothelial growth factor (VEGF), due to its proven proangiogenic nature, coupled with the fact that VEGF is the only proangiogenic factor known to act specifically on endothelial cells. It may be possible to promote rapid angiogenesis at the site where the new tissue is engineered by modulating the local concentration of VEGF over time. In essence, with this approach we are attempting to regulate how a specific cell type (endothelial cells) interfaces with a tissue engineering matrix with the goal of promoting this interaction to cause the formation of new blood vessels. According to the embodiment, the VEGF is incorporated in an alginate material in the manner described above and using the alginate or modified alginate materials described above.

The resulting alginate beads can be utilized to deliver the proangiogenic molecule VEGF in a rate-controlled and biologically active form. The gelling conditions regulate the incorporation and release of VEGF. Surprisingly, the biological activity of VEGF is enhanced considerably by incorporation in alginate. Desirable release kinetics can also be obtained by lyophilization of the alginate beads in the manner described above, which additionally makes them suitable for long-term storage. This system for drug delivery has potential applications for encouraging the rapid vascularization of engineered tissues. The VEGF will operate at the interface between the engineered matrix and the transplanted cells and encourage blood vessel invasion of the matrices. Such an effect facilitates providing the necessary nutrients to the transplanted cells so that a tissue of substantial size may be engineered.

Sustained release of growth factors has been demonstrated in a number of previous studies utilizing alginate and other polymer materials (Edelman et al., Biomaterials 12:619-626 (1991); Nugent et al., supra; Downs et al., supra; Gombotz et al., Bioconjugate Chemistry 6:332-351 (1995)). However previous studies utilizing alginate have generally focused either on the kinetics of release that can be achieved or the biological activity of the released growth factor, but both issues have not been thoroughly addressed in the same study.

Other microsphere controlled release systems for these types of factors include copolymers of lactic and glycolic acid (PLGA) (Mooney et al.,supra). A potential drawback to the PLGA microsphere systems is the large burst release typically obtained with these systems unless special processing conditions are utilized (Pekarek et al., Nature 367:258-260 (1994); Cleland, Pharm. Biotechnol., 10:1-43 (1997)). A large burst release may increase the local drug concentration to an undesirably high level or simply lead to the loss of the excess drug. Alginate, when gelled under appropriate conditions, has been demonstrated in the present study to provide nearly constant release (zero-order kinetics) after a minimal (8%) initial burst. Constant release is often desirable in order to maintain the released drug at a constant optimally effective concentration.

It is important to note that the solution in which the beads were placed had a significant effect on the release kinetics. Alginate beads in sterile water released over 50% of the incorporated VEGF in the first 24 hours, and the beads themselves began to break apart after 3 days. Therefore, all release studies reported in this paper were performed in tissue culture medium to more closely mimic the in vivo environment.

Quantification of the total VEGF incorporated in varying processing conditions reveals that the presence of SHMP, a gelling retardant, or heparin sepharose beads increases the amount of VEGF that is retained in the beads during formation. It is not surprising that heparin sepharose beads greatly enhance incorporation, as previous studies demonstrate that they act as a heparin depot to provide adhesion sites for heparin binding molecules such as VEGF (Edelman and Nugent, supra). The increased incorporation resulting from the addition of SHMP was unexpected. One would expect that slowing the bead formation would allow more VEGF to diffuse away from the alginate solution into the surrounding CaCl₂ solution during the gelling process. However, the opposite result was found. We hypothesize that the negative charge of alginate and the positive charge of VEGF lead to ionic interactions. The presence of SHMP lengthens the time that charged sites on the alginate are available for binding the VEGF before they bind to Ca²⁺ and perhaps become unavailable. This hypothesis is supported by the finding that higher calcium concentrations in the gelling solutions resulted in a more rapid release of VEGF from the alginate. The higher calcium concentrations may have the opposite effect from the SHMP by speeding gelling and preventing an association between the alginate and VEGF.

The biological activity of VEGF incorporated into alginate was equivalent to approximately four times the activity of the same amount of VEGF not incorporated into alginate. Similar results to this have been observed by other laboratories working with heparin-binding growth factors and alginate, although this enhancement was not quantified in past studies (Downs, supra; Nugent, supra; and Kawada et al., FEBS Letters, 408:43-46 (1997)). The recorded bioactivity of VEGF released during the first 15 days is greater than that expected from the total VEGF incorporated into the alginate beads. This suggests that VEGF activity is being enhanced through its interaction with alginate, perhaps via an ionic interaction that stabilizes this growth factor, since the half-life of VEGF has been measured to be 2-3 hours in the presence of HUVEC (Bikfalvi et al., J. Cell. Physiol., 149:50-9 (1991)). It is possible that the interaction of VEGF with alginate prolongs its biological half-life by shielding the VEGF from environmental conditions that would normally promote denaturation. A second possibility is that the alginate serves a metering function for the VEGF by delivering the molecules slowly over time rather than allowing the VEGF to immediately bind to any available cellular receptor.

The beads are observed to slowly fragment over time, and alginate dissolving from the beads may be altering the stability or binding of the VEGF to cells. The enhancement of bioactivity seen in these studies is not seen with other drug release systems such as PLGA microspheres. Indeed, decreased bioactivity of released growth factors is more typical with these non-alginate systems (Eiselt et al., Biotech. Prog., 14:134-140 (1997); and Mooney (1996), supra).

There are unique benefits in the invention. One of the advantages is the simplification of generation of defined media and allowing for the tight control of GF concentrations within a medium. Furthermore our data demonstrate that one may be able to use significantly less GF (⅓-⅕ of normal) than needed when GF is added directly to medium formulations. This system will be beneficial to those in need of specialty media as well as to the standard cell culture laboratories. The demand for specialty mediums is projected to rise significantly in the next few years, especially as more and more investigators are moving away from cell culture products containing bovine or other animal products. The invention will prove especially desirable in the realm of cell culture for human use where cells are cultivated for future implantation into humans. Already legislation is under consideration to limit the use of cells for human applications that have been in contact with animal serum products.

The entire disclosure of all applications, patents and publications, cited above and below, is hereby incorporated by reference.

### BRIEF DESCRIPTION OF THE DRAWINGS

Various other features and attendant advantages of the present invention will be more fully appreciated as the same becomes better understood when considered in conjunction with the accompanying drawings wherein:

FIG. 1 shows a process for preparation of drug-containing alginate beads. The drug, i.e. growth agent, is added to a syringe containing 1 ml of ungelled alginate. The solution is vortexed to evenly distribute the agent and then dripped into 30 ml of a CaCl₂ solution. After allowing the beads to gel for 10 minutes the calcium solution is removed via vacuum filtration and the beads are collected.

FIG. 2 shows cumulative VEGF release from 2% alginate beads gelled in either 0.1 M CaCl₂ (□), or 1.0 M CaCl₂ (▪) as a function of time. The release was normalized to the total VEGF contained in the alginate beads.

FIG. 3 shows cumulative VEGF release from 2% alginate beads containing VEGF (□), VEGF+SHMP (▪), VEGF+HS (O), and VEGF+HS+SHMP (+). Beads were gelled in a 0.1M CaCl₂ solution, and the cumulative release was normalized to the total VEGF incorporated into the beads in each condition. Values represent mean and standard deviation (n=3).

FIG. 4 shows typical dose response of endothelial cell growth as a function of VEGF concentration (a), and biological activity of VEGF released from alginate beads (b). (a) Endothelial cell growth (represented as fractional increase of cell number from day 1 to 3) increased with the concentration of control VEGF in the medium. Values represent mean and standard error (n=4). (b) The biological activity of VEGF (measured using endothelial cell proliferation) released from alginate beads as a percentage of the activity of control VEGF (never incorporated into alginate). The biological activity was determined for the VEGF released at various time. Values represent mean and standard error (n=4).

FIG. 5 shows cumulative release of VEGF from lyophilized beads (”) and non-lyophilized beads (+) as a function of time following immersion in medium. Values represent the mean and standard deviation (n=3), and are normalized to the total VEGF incorporated into beads.

FIG. 6 shows the release kinetics of bFGF from 2% alginate lyophilized beads.

FIG. 7 provides a growth study showing that the material released from the alginate beads is functional for cell growth.

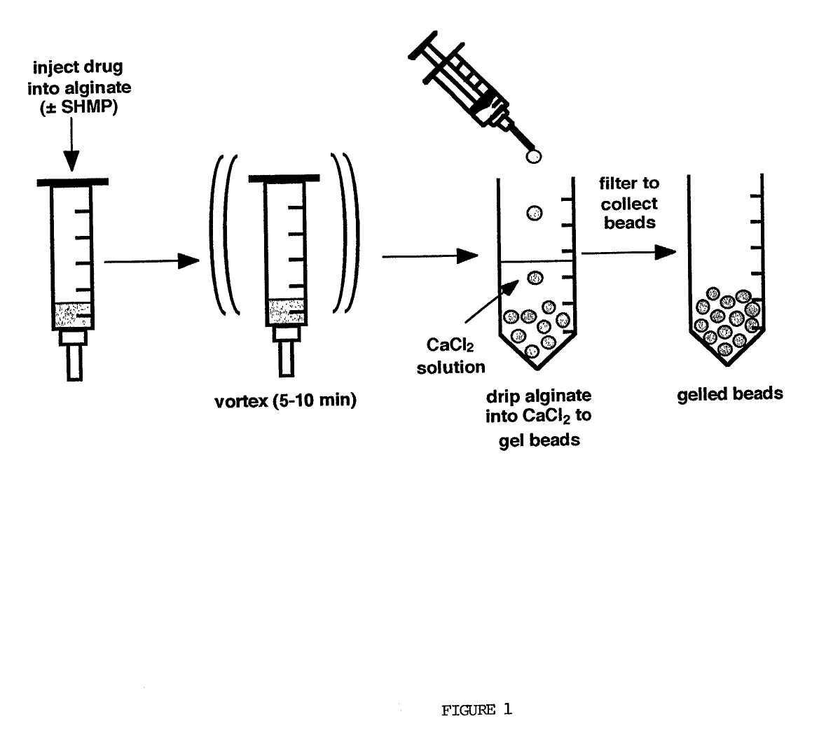

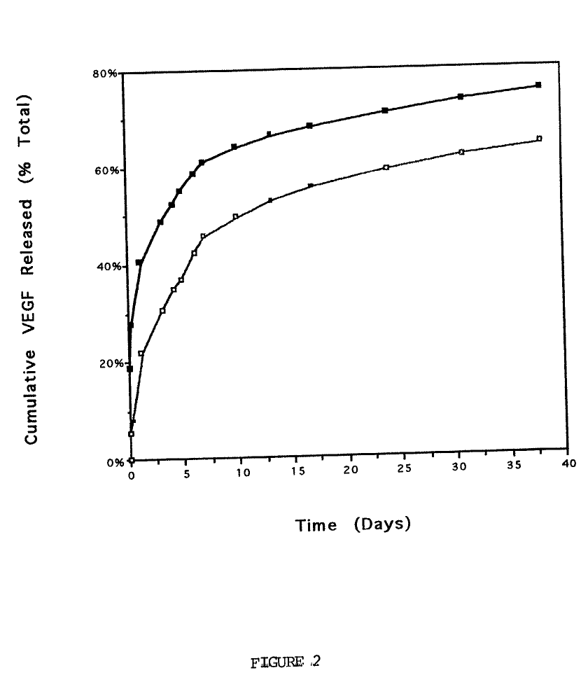

### EXAMPLES

In the foregoing and in the following examples, all temperatures are set forth uncorrected in degrees Celsius; and, unless otherwise indicated, all parts and percentages are by weight.

### Alginate Bead Preparation, Characterization and Properties

A 2% (w/v) high mannuronic acid sodium alginate (Pronova MVM; Norway) solution was prepared both with and without 0.2% (w/v) sodium hexametaphosphate (SHMP)(Alpha; Ward Hill, Mass.) in ddH₂O. Approximately 1.5 μg of VEGF (Intergen; Purchase, N.Y.), dissolved in 50 μL of ddH₂O, was then combined with 1 ml of the alginate solution and vortexed for 5-10 minutes to ensure adequate mixing. The mixed solution was then dripped, using a syringe with an 18 ga. needle, into 30 ml of a 1.0-0.1 M CaCl₂ (Sigma; St. Louis, Mo.) aqueous solution to yield approximately 50 beads. The resulting alginate beads were allowed to gel in the calcium solution for 10 minutes after which time they were collected by removing the aqueous solution via vacuum filtration. To determine the size distribution of the alginate beads, 30 individual beads were measured with vernier calipers.

To determine the efficiency of VEGF incorporation and the kinetics of VEGF release from the beads, receptor grade ¹²⁵I-labeled human VEGF (100 μCi/μg; Biomedical Tech. Inc., Stoughton, Mass.) was utilized as a tracer. This tracer, designed for radio-immuno assays, demonstrates stability and biological activity similar to unlabeled VEGF (company literature, Biomedical Tech. Inc.). Approximately 1 μCi of labeled VEGF was added to a 50 μL aqueous bovine serum albumin fraction V solution (BSA, 10 mg/ml; Gibco; Grand Island, N.Y.) which was then injected into 1 ml of the alginate solution. For the tracer experiments this BSA solution was used in place of the VEGF/H₂O solution mentioned above. The beads were prepared as previously described. After bead fabrication, the radioactivity in a known number of beads was counted in a gamma counter and the incorporated activity was compared to that of the initial aqueous VEGF/BSA solution to calculate the percentage of the total VEGF that was incorporated into the beads. To determine the release of VEGF from the beads a known number of beads (15-20) prepared with the radio-labeled VEGF were placed in a known volume (5 ml) of glucose free Dulbecco's Minimum Essential Medium (DMEM, Gibco) and placed in an incubator maintained at 37° C. At set times, samples (4.5 ml) of the DMEM release media were collected, and the removed volume was replaced with fresh media. The amount of ¹²⁵I-VEGF released from the beads was determined (n=3) at each time point by counting the removed samples in a gamma counter and comparing the result to the total ¹²⁵I-VEGF loaded into the beads.

In several release kinetics experiments heparin sepharose microspheres (55 mg/ml alginate, CL-6B; Pharmacia Biotechnology Inc.; Piscataway, N.J.) were added to the alginate along with the labeled VEGF.

Alginate beads with incorporated VEGF exhibited a spherical shape, with an average diameter of 3.3±0.1 mm. The CaCl₂ concentration was varied in the first set of experiments to determine if the extent of calcium cross-linking would regulate the release of incorporated VEGF. The efficiency of VEGF incorporation into 0.1 M CaCl₂ cross-linked beads was 31±1% while beads gelled in a 1.0 M CaCl₂ solution demonstrated approximately 20% incorporation.

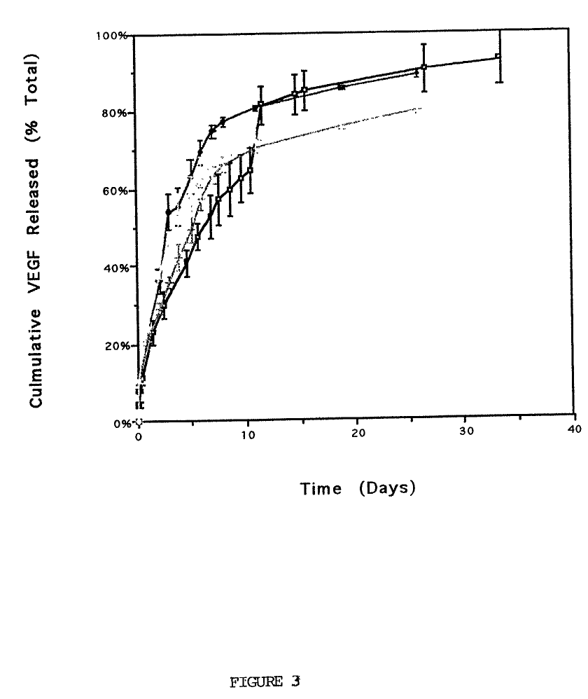

The VEGF release properties for these beads are shown in FIG. 2. Alginate beads gelled in a 0.1 M CaCl₂ solution (shown in the lighter shaded line) exhibited a minimal burst of VEGF (8.1% in 2 hours), and nearly zero-order release (5.3%/day) for the ensuing 7 days. The release slowed to 0.6%/day after this time, and this release rate was maintained for the duration of the experiment. The burst observed from the beads gelled with 1.0 M CaCl₂ (the darker line) was much greater (27.7% in 2 hours and a total of 40% in the first day) with a constant release (3.6%/day) continuing for 5 days following the burst. After 6 days the release leveled off (0.5%/day) for the remainder of the experiment.

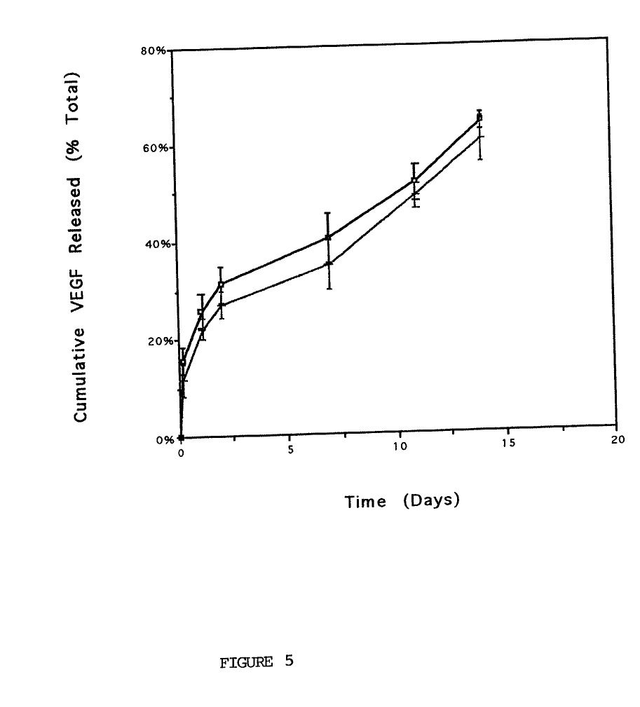

For the data shown in FIGS. 3-6 the alginate beads were gelled in 0.1 M CaCl₂, due to the favorable release kinetics, and allowed to release into DMEM.

The effects of providing both a heparin depot (heparin sepharose beads) for VEGF within the alginate beads and a retardant for alginate gelling on the incorporation and subsequent release of incorporated VEGF was assessed. Beads prepared with and without both heparin sepharose beads and SHMP were analyzed for the percentage of the radioactive tracer incorporated into the beads as follows:

TABLE 1
VEGF Incorporation

| Condition | Percent Incorporation |
|---|---|
| VEGF | 31 ± 1 |
| VEGF + SHMP | 37 ± 1 |
| VEGF + HS | 64 ± 1 |
| VEGF + HS + | 65 ± 2 |
| SHMP | |

VEGF: vascular endothelial growth factor
SHMP: Sodium hexametaphosphate
HS: Heparin sepharose spheres

Bead formation in the presence of SHMP slightly improved VEGF incorporation, while bead formation with heparin sepharose beads approximately doubled the VEGF incorporation efficiency. When the VEGF containing beads were placed in tissue culture media zero-order release kinetics (approximately 5%/day) were observed in all samples for the first 14 days with a diminished steady release (0.75%/day) for an additional 14 days. See FIG. 3 wherein the darkest line represents VEGF alone, the second darkest line with solid points represents VEGF with SHMP, the third darkest line with the crossed points represents VEGF with SHMP and HS and the lightest line with the open circle points represents VEGF with HS.

### In Vitro Analysis of the Biological Activity of Released VEGF

The biological activity of VEGF incorporated into, and released from, alginate beads was determined by testing its ability to stimulate the growth of cultured human umbilical vein endothelial cells (HUVEC, passage 14-15; American Type Culture Collection; Rockville, Md.). In brief, a known volume (5 ml) of medium was incubated with a known number (12-15) of VEGF releasing alginate beads. Samples (4.5 ml) were collected at predetermined times, and the removed volume was replaced with fresh media. The VEGF content of collected samples was calculated using the known release kinetics determined using ¹²⁵I-labeled VEGF tracer. The biological activity of these samples was then analyzed with a cell proliferation assay. Briefly, HUVEC were plated at a density of 5000 cells/cm² on 24 well tissue culture dishes (Corning; Cambridge, Mass.). Cells were refed at 24 hours with media containing known amounts of VEGF released from alginate beads, or with media that had a known concentration of soluble VEGF (0-40 ng/ml). This latter VEGF, which was never incorporated into alginate, served as a control. The cells were then placed in an incubator at 37° C. for another 48 hours. Following this incubation the number of cells in each well was quantified by removing the cells with a solution of 0.05% trypsin/0.53 mM EDTA (Gibco), and counting them in a coulter counter. The biological activity of VEGF released from alginate beads was determined by comparison to the stimulatory effect observed in the culture wells containing control VEGF. Serum and mitogen free media (Cell Systems; Kirkland, Wash.) supplemented with 10% fetal bovine serum (Gibco) and 50 μg/ml gentamicin (Gibco) was used for all experiments. To increase cell plating efficiency 20 ng/ml of VEGF was added to the media used to plate cells. This media was removed, and replaced with the media samples described above at the start of the experiment.

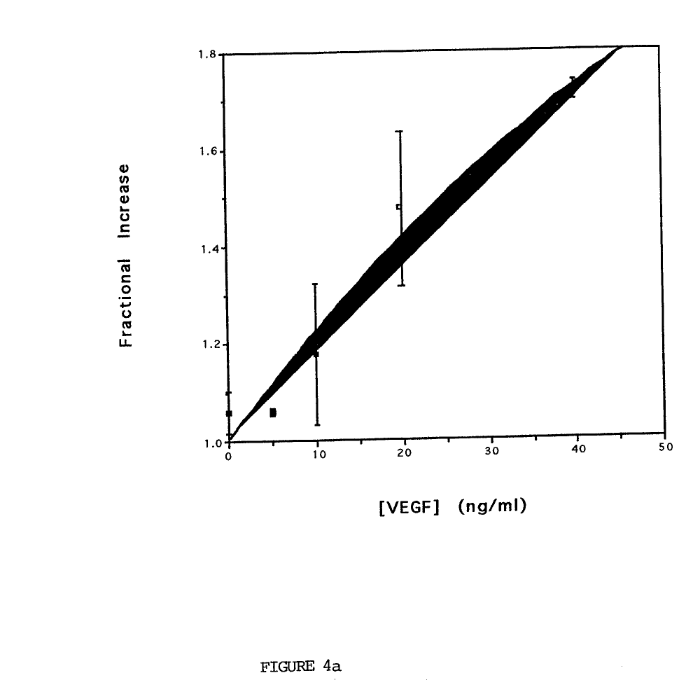

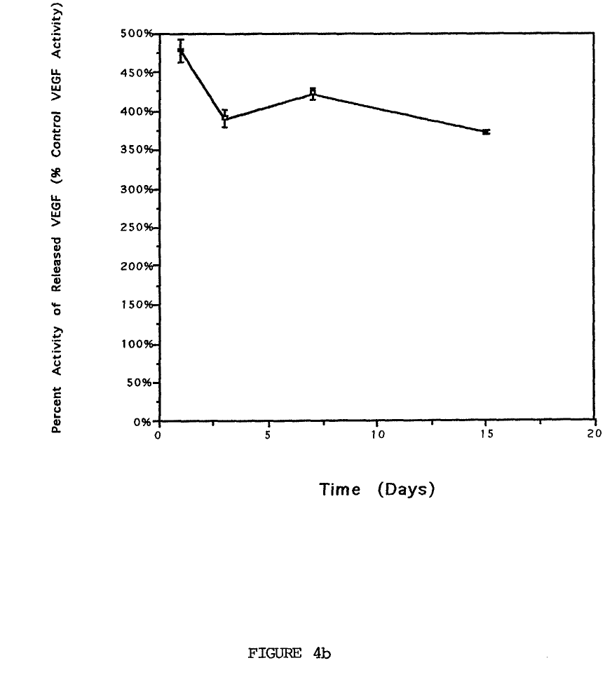

VEGF normally stimulated endothelial cell growth in a dose-dependent fashion when added to tissue culture medium (FIG. 4a). The released VEGF was also found to stimulate endothelial cell growth and, by comparing the stimulatory effect of released VEGF to control VEGF, a value for the biological activity of the released VEGF could be determined. Surprisingly, the activity of the VEGF released from the alginate beads was greatly enhanced, as it was approximately three to five times as effective as the control VEGF (FIG. 4b).

### Release Kinetics of Lyophilized Alginate Beads Containing VEGF

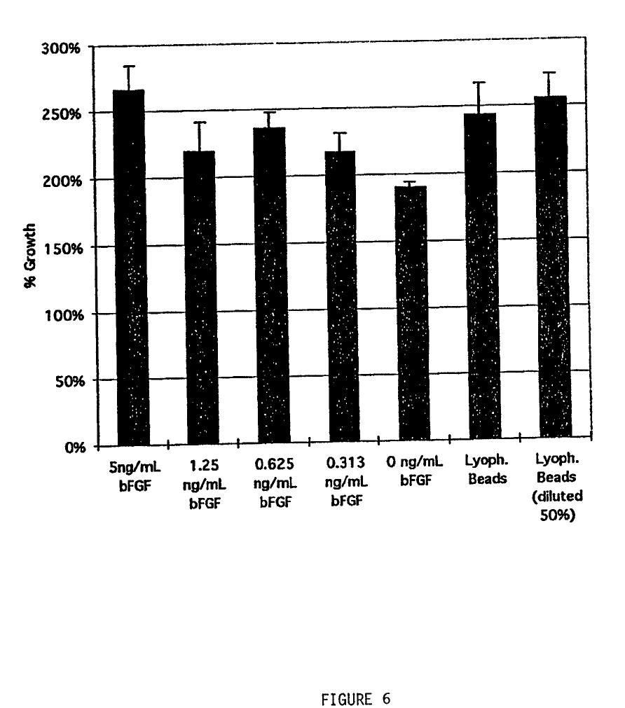

To determine if the processed alginate beads could be stored without changing the desirable VEGF release kinetics, several samples of the above-described beads were lyophilized. The VEGF release kinetics from these beads was then determined following their placement in medium. The dried beads were much smaller initially but fully rehydrated within 24 hours. The kinetics of release from the lyophilized alginate beads was largely unchanged from that demonstrated by non-lyophilized beads. See FIG. 6 wherein the darker line with the open square points are lyophilized beads and the lighter line with the crossed points are non-lyophilized.

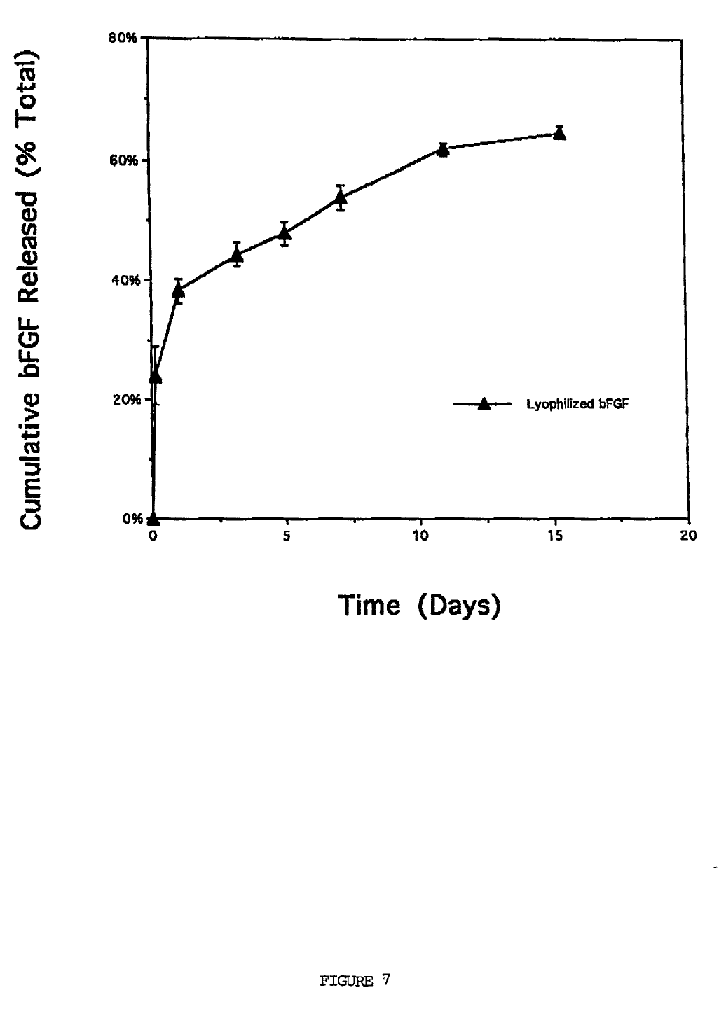

To further confirm that biologically active GF could be released from beads after lyophilization, basic fibroblast growth factor (bFGF) was incorporated into (in amount of about 70%), and released from, lyophilized alginate beads. The biological activity of the released factors was determined by testing its ability to stimulate the growth of cultured rat aortic smooth muscle cells (SMC). Minimum essential medium supplemented with 2% fetal bovine serum and was used for all experiments. Varying amounts of soluble bFGF, not incorporated into beads, were added to the medium in additional experiments. Medium with no bFGF was incubated with bFGF releasing alginate beads for 2 weeks with samples (4.5 mL) being collected at predetermined times. After each sample collection the removed volume was replaced with fresh medium. The bFGF content of collected samples was determined using the known release kinetics (FIG. 6). The samples were then analyzed with a cell proliferation assay. Briefly, SMC were plated at a density of 5000 cells/cm² on 24 well tissue culture dishes. After allowing the cells to attach for 24 hours the wells were refed with either media containing known amounts of bFGF released from alginate beads or media with a known concentration of soluble bFGF never incorporated into alginate (0-5 ng/mL, control). The cells were then placed in an incubator at 37° C. for another 48 hours. Following this incubation the number of cells in each well was quantified by removing the cells with a solution of 0.05% trypsin/0.53 mM EDTA, and counting them in a coulter counter. The biological activity of bFGF released from alginate beads was determined by comparison to the stimulatory effect of control bFGF (FIG. 7). The data indicate that diluting the samples had no effect on bioactivity suggesting the presence of more then double saturating concentration of the released growth factor.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. Described herein are materials and methods for the controlled delivery of growth factors and other materials which facilitate the growth of cells.

We claim:

1. A medium for cell culturing which comprises a cell growth agent contained within a polymeric structure which provides controlled release of the cell growth agent and at least one other component needed for cell culturing.

2. The medium of claim 1, wherein the polymeric structure is an alginate or modified alginate.

3. The medium of claim 1, wherein the polymeric structure has sufficient porosity characteristics and/or degradeability to provide a controlled release period of the cell growth agent of from 1 day to 3 months.

4. The medium of claim 1, wherein the at least one other component includes a nutrient for cell growth.

5. The medium of claim 1, wherein the medium contains no serum component.

6. The medium of claim 1, wherein the cell growth agent is a cell growth factor, a hormone or another lipo-protein or glyco-protein.

7. The medium of claim 1, wherein the cell growth agent is a vascular endothelial growth factor, a basic fibroblast growth factor or an epidermal growth factor.

8. The medium of claim 1, wherein the cell growth agent contained within a polymeric structure is in the form of spherical beads having an average diameter of from 1 mm to 5 mm.

9. The medium of claim 8, wherein the beads have been lyophilized before incorporation into the medium.

10. A method for culturing cells in a medium, which comprises adding to the medium particles of a polymeric structure containing a cell growth agent which is controllably releasable therefrom.

11. Lyophilized particles of a polymeric structure containing a cell growth agent which is controllably releasable therefrom.

12. The lyophilized particles of claim 11, wherein the polymeric structure is an alginate or modified alginate.

13. The lyophilized particles of claim 11, wherein the cell growth agent is a cell growth factor, a hormone or another lipo-protein or glyco-protein.

14. The lyophilized particles of claim 11, wherein the cell growth agent is a vascular endothelial growth factor, a basic fibroblast growth factor or an epidermal growth factor.

15. The lyophilized particles of claim 11, wherein the polymeric structure has sufficient porosity characteristics and/or degradeability, when reconstituted, to provide a controlled release period of the cell growth agent of from 1 day to 3 months.

16. The lyophilized particles of claim 11, which has storage stability of up to 6 months with no significant loss of activity in providing a controlled release of active cell growth agent.

17. The lyophilized particles of claim 11, having an average diameter of from 10 nm to 1 mm.

18. The lyophilized particles of claim 11, having an average diameter of from 1 mm to 5 mm.

19. A method for culturing cells in a medium, which comprises adding to the medium lyophilized particles according to claim 11.

20. A method for tissue engineering, which comprises using the lyophilized particles according to claim 11 for seeding along with cells onto a polymer matrix for tissue transplantation, by incorporating them directly into the bulk of a matrix for tissue engineering or implanting them in vivo for tissue generation.

21. Particles of an alginate or modified alginate containing vascular endothelial growth factor such that the factor is controllably releasable therefrom.

22. The particles of claim 21, having an average diameter of from 10 nm to 1 mm.

23. The particles of claim 21, having an average diameter of from 1 mm to 5 mm.

24. The particles of claim 21 in lyophilized form.

25. A method for enhancing vascularization of a tissue engineering matrix which comprises using the particles according to claim 21 for seeding along with cells onto the matrix or by incorporating them directly into the bulk of the matrix.
